(19) United States
(12) Patent Application Publication
(10) Pub. No.: US 2012/0015449 A1
(43) Pub. Date: Jan. 19, 2012

(54) METHOD AND DEVICE FOR DETERMINING A FOREIGN SUBSTANCE CONTENT IN A MATRIX

(75) Inventor: Andreas Fiedler, Rottenburg a. N. (DE)

(73) Assignee: CRINOTEC GMBH, Tubingen (DE)

(21) Appl. No.: 13/256,873

(22) PCT Filed: Dec. 14, 2009
(86) PCT No.: PCT/EP2009/008936

§ 371 Date: Sep. 15, 2011

(30) Foreign Application Priority Data
Mar. 19, 2009 (DE) 10 2009 013 534.

(51) Int. Cl. G01N 33/02; G01N 27/00; B01L 3/00; G01N 33/14; G01N 21/75

(52) U.S. Cl. 436/501; 436/20; 422/82.05; 422/547; 422/69; 422/82.01; 422/430

(57) ABSTRACT

A method and a device are provided for determining a content of at least one foreign substance in a matrix of a solid or liquid food. At least one reagent area for providing at least one reagent has a receptacle for a replaceable reagent container for replaceably connecting a sample container including at least the matrix; a transfer line is provided between the reagent area and the reaction area, by which the at least one reagent can be fed to the reaction area; a sensor area is provided for demonstrating the foreign substance released from the matrix; a carrier gas line is provided between the reaction area and the sensor area, by which the released foreign substance can be fed to the sensor area; and an output line is provided through which the dissolved foreign substance can be fed outward from the sensor area.

### BACKGROUND AND SUMMARY

The invention relates to a method and device for determining the content of a foreign substance in a matrix according to the preamble of the independent claims.

It is of interest in various fields to know the content of a foreign substance that is in a matrix, for instance a liquid sample, and that is free and/or bound to constituents. One such component is, for example, sulphur dioxide (SO₂), the content of which is to be determined, for instance, in wine. The presence of SO₂ in fruit juices or other foods, including those of solid consistency, also cannot be neglected for health reasons.

Furthermore, SO₂ is of importance in environmental analysis, for example in control systems for exhaust air, flue gas desulfurization and determination of the maximum permissible workplace concentration etc.

The legislator has now laid down maximum values for many areas of SO₂ usage which must be met by industry, producers and filling facilities and which consequently must be checked at regular intervals.

It is only possible to do without SO₂, for example in wine production, to a certain degree, because SO₂ is used, for example, to treat the wine barrels, and also serves as a preservative and is generally regarded as an indispensible component for high-quality wine production. Nevertheless, SO₂ has adverse health effects that manifest themselves, for example, through severe headaches after drinking wine with high concentrations of SO₂.

In addition, for wine in particular there are a large range of wine faults and wine diseases which are caused by unwanted or excessive concentrations of components.

This includes, for example, the odour caused by the presence of acetic acid, “Boeckser” aroma caused, inter alia, by hydrogen sulphide, the cork taint caused by 2,4,6-trichloroanisole and the woody taint caused by 3-methyl octano-4-lactone.

For further information on components causing wine faults and wine diseases see Römpp Lexikon Chemie [Römpp's Lexicon of Chemistry], 10th Edition, 1999, Georg Thieme Verlag, Stuttgart.

Sulphur dioxide/sulphurous acid is present in wine, both free and bound to various constituents. The total content of sulphurous acid is represented by the sum of all state forms, which must be monitored in terms of certain maximum values. The presence of bound SO₂ is mainly due to the so-called bisulphite addition to aldehydes and ketones.

A whole range of measurement methods are known to determine free sulphuric acid or free SO₂ from statutory analysis specifications and from corresponding manuals for wine growers. A distinction is drawn between so-called reference methods and rapid methods. The known reference methods are very time consuming, require complex apparatus and are correspondingly expensive. Analyses using rapid methods are inaccurate and are poorly reproducible because of numerous interference parameters. One problem is that ascorbic acid is very often present in wine and, for example, is incorrectly identified as sulphurous acid in iodometric methods, which are classed as a titrimetric method and are amongst the recognised rapid methods. To determine the concentration of free sulphurous acid, it is necessary to determine the concentration of ascorbic acid separately and to subtract this from the value obtained.

In addition to iodometry it is also known to determine SO₂, for example, using colorimetry, oxidation methods, gas measuring electrodes, enzymatic methods or through gas chromatography.

To allow bound sulphurous acid to be determined by the known methods it is necessary to release it through alkaline saponification or by heat through a recognized reference method such as distillation with strong acids, which necessitates additional, costly and time-consuming process steps which, moreover, are not always quantitatively sufficiently reliable.

In gas chromatography, for example, using a flame photometer or an electrolytic Hall detector, the SO₂ concentration is measured in the headspace above the liquid sample, then Henry's law is applied to calculate the concentration of sulphurous acid in solution. Henry's Law, also known as Henry's law of absorption, states that the vapour pressure of a dissolved substance is proportional to its mole fraction in an ideal diluted solution. The proportionality is expressed by an empirical, temperature-dependent Henry's constant. Although the measurement with subsequent calculation results in a satisfactory outcome, the use of a Hall detector or a flame photometer is, however, limiting for the method given. Both the cost and the required experimental experience for the use of the specified detectors limit its application to large specialist facilities. For smaller wineries, bottlers or analytical laboratories, the use of the known method is not viable.

The procedures described so far for sulphur dioxide analysis thus require a substantial investment of time and equipment expense, and are particularly disadvantageous in that other components are also determined at the same time, so that these must be measured separately and subtracted from the obtained measured value.

A simplified device and a method for measuring the content of SO₂ in wine is known from EP 1285 266 B1, in which the wine is aerated by a carrier gas that is passed in a short circuit through the wine. This gives rise to an equilibrium state so that the concentration of SO₂ present in the gas phase can be detected and evaluated by means of an electrochemical cell. Gas feed can be automated during sample preparation. The method delivers sufficiently good results. A device is known from EP 1 840 557 with which the composition of liquids can be determined. Reagents are introduced into a reagent area and the reagents added with metering to a sample in a reaction area, which liberates constituents in a head space or forms a volatile compound with them. The composition can be determined qualitatively and quantitatively in that absorption spectra of the gas in the head space are recorded and the absorption behaviour of the gas is analysed.

U.S. Pat. No. 3,873,273 A discloses an automated system for testing blood samples. The presence of blood samples is established and optical transmission properties of the blood samples, to which a reagent is added, are investigated.

WO 83/00932 discloses an apparatus for the storage and delivery of reagents. Reagent containers are attached to container receptacles of a carrier, wherein during the attachment process a needle penetrates a pierceable septum in the container receptacle.

The aim of the present invention is to create a further improved method and device with which less experienced people can also detect the presence of a foreign substance in a matrix on site.

This aim is achieved through the features of the independent claims. The other claims, the drawings and description describe favourable embodiments of the invention.

A device is proposed for the determination of the content of at least one foreign substance in a matrix of a solid or liquid food, that has at least one reagent area for provision of at least one reagent with a receptacle for an interchangeable reagent container, a reaction area with a connection system for connecting the sample container at least containing the matrix, a transfer line between the reagent area and the reaction area, with which the at least one reagent can be transferred to the reaction area, and a sensor area for the detection of the foreign substance released from the matrix, a carrier gas line between the reaction area and the sensor area, with which the released foreign substance can be transferred to the sensor area, and an output line through which the dissolved foreign substance can be transferred from the sensor area outwards.

The reagent container and/or sample containers are advantageously prefabricated and already pre-filled with chemical components that are exactly matched to one another in terms of nature and quantity. Particularly preferably, they are configured so that they can be used in the device without the danger of mix-ups. The user only needs to introduce the matrix to be investigated, possibly containing a foreign substance, in a pre-determined quantity. For example, a vintner can check the sulphite (SO₂) content of the wine in the wine cellar. SO₂ in this case represents the foreign substance and wine is the matrix. However, it is also possible to analyse sulphur-treated dried fruit that is broken into small pieces and mixed, for example, with water, and to analyse fruit juices, beer and the like. Food is to be understood here as substances and products that humans ingest for the purpose of nutrition or enjoyment by mouth, possibly after prior preparation. This in general includes food, beverages, food additives and food supplements. Similarly, feedstuffs for animals can also be subsumed under it.

Hereinafter, SO₂ refers to the sulphite content, which may be present in dissolved form or bound in the matrix. Dissolved SO₂ is also referred to as free SO₂. The device can advantageously be used for the detection of free SO₂ alone and for the detection of the total content of SO₂, which is present in the food in dissolved and bound form.

The reagent area may advantageously comprise at least one sensor device that automatically detects a presence or absence of the at least one reagent and/or the presence of a reagent container in the reagent area. Similarly, the type of reagent in the reagent container can additionally be detected. The sensor device can, for example, be a light barrier that detects a coding on the reagent container, a barcode, an RFID encoding (Radio Frequency IDentification), a lever, a micro switch and the like.

The reagent area can advantageously have a receptacle for receiving a reagent container, preferably, the reagent container can be fully contained in the receptacle. The reagent container can be inserted in a carrier housing and with the carrier housing can be inserted in the reagent area. As a result, the reagent container can be handled more easily and together with the carrier housing can be removed easily from the receptacle. The carrier housing can be configured to hermetically seal the receptacle, which increases the safety of the device. The reagent container can, for example, be configured as a pierceable ampoule with a pierceable septum as a closure, or be sealed with a welded-on or glued-on metal or plastic film.

The reagent area can have a needle system for attachment of the reagent container, onto which the reagent container can be attached. The needle system can preferably comprise at least two hollow needles, which are immersed in the reagent container. One needle preferably represents the inlet to the reagent container, which can be connected, for example, to a transport device, for example a pump or reservoir, the other represents an outlet, which can be connected, for example, to the reaction area. The needles can also extend to different depths into the reagent container. The longer needle is first to come into contact with the interior of the reagent container. The longer needle preferably represents the outlet for the reagent. As a result, any excess pressure in the reagent container upon connection of the reagent container can reliably transport the reagent in the desired direction to the reaction area, if this is connected via the longer needle to the reagent area. The needle system can comprise needles arranged in parallel, or the needles can be arranged coaxially, for example a shorter needle can be inserted into a longer needle. Alternatively, a hollow needle with an inner hose can also be provided.

Additionally or alternatively, a check valve can be provided on the input side, thereby ensuring that the reagent can only leave the reagent container in a desired direction, even if the needles extend the same distance into the reagent container. In addition, the needles can be advantageously configured as a modular unit so that the needles can be readily exchanged. For this, the needles can be integrated into a block that has the appropriate connection possibilities for a transport means and for a connection to the reagent area.

The reaction area can advantageously have a feed system for connection of a sample container through which at least the reagent can be fed. The released foreign substance can preferably be dischargeable through the feed system and, for example, be passed to the sensor. The feed system can be configured as a needle system. It is also conceivable, however, to provide hoses or small tubes. For this, a parallel arrangement of needles and/or hoses and/or tubes lying next to one another, as well as a coaxial arrangement, is possible. The feed system can preferably be guided through a stopper, which can serve as splash protection and can also seal the sample container when it is connected to the reaction area. The sample container is preferably attached to a cone-shaped pin, from which the feed system with the plug projects. A sealing ring or a sealing lip for sealing of the attached sample container can be disposed on the pin. This allows the sample containers to be readily and reliably docked to the reaction area.

A sensor is advantageously introducible into a headspace of the sample container. This can be provided alternatively, or additionally, to a sensor in a sensor region at a distance from the sample container.

The reaction area can have a heating and/or cooling device. This allows the detection reaction to be advantageously suitably supported. The heating can be achieved, for example through external components such as a heating coil in the sample, heating of the line system, heating through radiation (for example infrared, microwaves), a heating jacket around the sample container, addition of substances that release heat upon dissolution etc. Similarly, in addition or alternatively, a use of reagents in the sample container is possible which because of their aggressiveness do not require additional heating, or which themselves release heat upon contact with a substance or the sample. Cooling can be carried out by external components or through the addition of substances that consume heat upon dissolution. An uncooled reaction is also conceivable.

The sensor area can preferably comprise an electrochemical sensor. The electrochemical sensor favourably comprises a reference electrode with a chloride-free redox system. The reference electrode preferably comprises a Pb/PbSO4 system. This allows a reference potential to be made available for the electrochemical sensor, to which the measured potential can be compared. The reference electrode system is favourably free of chloride and the system has a long-term stability of at least one year when installed. The sensor allows a simple and non-critical handling and is in particular stable in an acidic environment. There is in particular no “bleeding” of the system through concentration differences. A disturbance or poisoning of the foreign substance-sensitive working electrode of the electrochemical sensor can be avoided. If, depending on the chemical system, it is intended to work in the alkaline range, then the reference electrode system can be appropriately selected.

Alternatively or additionally, the sensor area can comprise a photometric sensor and/or provide other conventional detection methods.

According to an advantageous development of the device a carrier gas line between the reaction area and sensor area can be divided into a first branch line for supplying carrier gas to the sensor area and a second branch line to bypass the sensor area. For substances, such as the reagent, which can dry out a membrane or a layer of the sensor that reacts on the substance, an excessive exposure of the membrane can be avoided without a detrimental effect on measurement accuracy. Furthermore, the flow properties can be optimized. Since the gas does not have to flow directly past the membrane, advantageous diffusion-controlled quasi-static conditions can develop at the sensor area/at the membrane. Fluctuations in the gas flow can thereby be compensated for, which has a positive effect on measurement accuracy. This is advantageous if the carrier gas has time-varying concentrations of the analyte so that the concentrations at the sensor vary over time.

According to another aspect of the invention an interchangeable reagent container for a device for determining a content of foreign substance in a matrix of a solid or liquid food is proposed, which is provided in ready-to-use form with a defined quantity of at least one reagent for use in the device and can be connected interchangeably to the receptacle of the reagent area. One or more codings can be provided to detect the presence in the device and/or the nature of the contained reagent. The at least one reagent can be an acid, preferably at least one acid selected from the group of sulphuric acid, phosphoric acid and hydrochloric acid.

Depending on the type of matrix and foreign substance to be investigated, other inorganic or organic acids and/or other concentration ranges of the acids may be favourable. An alkaline reagent is also conceivable if it is intended to work in the alkaline range. Consequently, for the detection of SO₂ in wine for example, an alkaline hydrolysis is also conceivable to split bisulphite adducts with subsequent expulsion of SO₂ in an acidic environment.

The reagent container can advantageously have a corrosion inhibitor added to it. Thus, for the detection of sulphite in wine, a 50-85% sulphuric acid mixture can be modified by iron (III) salts such that the hollow needles of the needle system in the reagent area are not chemically attacked or only to a negligible degree. In addition to iron (III) salts, other salts are suitable, such as those of copper, tungsten or molybdenum, for example,

Al₂(SO₄)₃

MnSO₄*1aq

ZnSO₄*7aq

FeSO₄*7aq

CoSO₄*7aq

H₃[P(Mo₃O₁₀)₄]*xH₂O

NiSO₄*6H₂O

SnCl₂*2H₂O

CuSO₄*5aq

Fe₂O₁₂S₃*x aq

Bi₂(CO₃)₃

Na₂MoO₄*2H₂O

Na₂WO₄*2H₂O

VOSO₄*5H₂O.

If a plurality of reagents are used in the device, such as in the determination of free SO₂ and bound SO₂ in wine, then the various reagents can be mixed with the inhibitor to maintain a defined concentration of inhibitor.

According to another aspect of the invention an interchangeable reagent container for a device for determining a content of foreign substance in a matrix of a solid or liquid food is proposed, and can be connected interchangeably to the connection system of the reaction area, wherein at least one member of the group is (a) an adduct former in a metered quantity that is matched to the foreign substance to be detected and which serves to bind any dissolved foreign substance in the matrix, (b) a substrate that dissolves in contact with a reagent and/or a matrix, and (c) a chemical component with positive enthalpy of solution, so that the chemical component absorbs heat upon being dissolved in the matrix.

The sample container is in particular matched in its dimensions to the device and the reagent and sample quantities used. Measurement errors due to incorrect dosages can thus be avoided.

One adduct former can favourably be contained in a metered quantity. The adduct former is present to bind any dissolved foreign substance present in the matrix. In this way previously dissolved, i.e., “free” portions of the foreign substance in the matrix and the previously chemically bound portions of the foreign substance in the matrix can be released later virtually simultaneously.

Favourable adduct formers are in particular molecules with aldehyde and/or ketonic functions, possibly also in ring form (e.g., sugar). Alternatively, adsorbents can be used which can bind the foreign substance to their surface. Alternatively, other substances can be used which bring about the retention of a foreign substance, for example an oily layer, polymerisation upon sample addition (wine addition) and/or addition of acid etc. Polymerisation can be achieved for example through condensation of benzyl alcohol or its esters with strong acid, i.e., addition of benzyl alcohol to the sample, whereby polymer formation commences upon introduction of the acid.

The adduct former can preferably be matched to the foreign substance to be determined, such as SO₂. For detection of SO₂, pyruvic acid, or a salt of pyruvic acid, preferably a sodium salt of pyruvic acid, is preferred as the adduct former.

The sample container can, additionally or alternatively, contain a substrate that comprises a substance that is capable of forming a network, in particular a three-dimensional network, such as agar or gelatine. The substrate can dissolve in contact with a reagent and/or a matrix and ensure mixing of reagent and adduct former. The substance can, for example, be a complex-forming polymer, a chelating agent or a gel. The substrate can be formed in particular by the substance. The skilled person will choose a suitable substrate depending on the specific boundary conditions and materials. By dissolving the substrate in contact with a reagent and/or a matrix, an advantageous mixing of the sample, i.e., matrix with foreign substance and the adduct former and/or the reagent can be achieved. The analytical times can thereby be advantageously reduced. Mixing can conceivably be carried out as an alternative or additionally through stirring, vibration and the like.

In addition or alternatively, a chemical component with a positive enthalpy of solution can advantageously be present in the container. This can bring about cooling of the sample container, in particular for the detection of free SO₂.

According to a further aspect of the invention a set is proposed, comprising a reagent container and a sample container for use in a device for determining the content of at least one foreign substance in a matrix of a solid or liquid food. The reagent container can advantageously contain an acid to release the bound foreign substance and the sample container can contain an adduct former that is matched to a foreign substance to be detected. The reagent container is filled ready for use with a suitable reagent to detect a foreign substance in a matrix. The sample container is similarly filled ready for use and can contain at least one member of the group

(a) an adduct former in a metered quantity that is matched to the foreign substance to be detected;
(b) a substrate that dissolves in contact with a reagent and/or a matrix;
(c) a chemical component with a positive enthalpy of solution. Optionally, heating can be achieved through a component with negative enthalpy of solution or reaction enthalpy.

A method is proposed for operation of a device for determining a content of at least one foreign substance in a matrix of a solid or liquid food, wherein an originally dissolved foreign substance is first bound in the reaction area in the matrix and the foreign substance originally dissolved in the matrix is released with a delay, such that the originally dissolved foreign substance and any foreign substance originally bound are released together from the matrix in the same process step. This is carried out in particular in the determination of a total content of SO₂, which is present in the matrix in dissolved and bound forms.

The matrix can advantageously be mixed with an adduct former for binding of the dissolved portion of foreign substance. After binding of the originally dissolved foreign substance a reagent can be added which drives out the originally dissolved foreign substance and any originally bound foreign substance from the matrix. The driven-out foreign substance can preferably be fed through a carrier gas to a sensor for the detection of the foreign substance.

Alternatively, it is also conceivable, instead of adding an adduct former, to cover the matrix, to hold back a foreign substance released early and to only release it when the portion of the foreign substance originally bound in the matrix is released. It is also conceivable to combine the addition of an adduct former and the covering of the matrix.

Since measurement, for example of the SO₂ content in wine is not carried out in chemical equilibrium, different time-dependent concentration profiles for different sulphate-containing species, bound to different extents, are to be expected. It has been found that sulphite present in the wine sample as free SO₂ exhibits a time-shifted forwardly displaced peak in the measurement curve for concentration, which falls relatively rapidly after reaching the maximum concentration. For species in which the chemical equilibrium lies very much on the side of the bound SO₂, curves can be seen in which the peak in the time-dependent

concentration profile is shifted in time strongly backwards and the fall after the maximum concentration is relatively slow. According to an aspect of the invention, with the delayed release of the originally unbound foreign substance from the form bound to the adduct former even in the presence of very different species in unknown concentration ratios it is possible to equalise the concentration profile for simple mathematical determination of the concentration via the sum of all species of the particular foreign substance, for example the sulphite species in wine, dried fruit, fruit juices, beer and the like.

In the detection of total foreign substance content in the matrix, which is present originally in dissolved and bound form, the foreign substance adduct can be transformed again into the educts (adduct former and foreign substance) through heat and acid influence. By means of a gas stream, the dissolved foreign substance, for example the dissolved SO₂, is transferred from the solution into the gas stream and passed through a foreign substance-sensitive sensor in which a concentration-dependent signal is generated.

Quantitative release of the bound foreign substance, for example the bound SO₂ in a matrix, e.g., in wine, requires drastic ambient conditions (high temperature, strong acid) or requires a long time period to achieve chemical equilibrium. Since the measurement is carried out according to an aspect of the invention by a gas stream that is passed through the sample, the quantitative conversion can proceed very rapidly.

The heating of the sample in the sample container can preferably be carried out through the resulting hydration energy/dilution heat that is released when concentrated acid comes into contact with an aqueous medium. Advantageous here is the elimination of mechanical and/or electrical components, as well as the possibility of a common line route for reagents to detect dissolved foreign substance (for example free SO₂ at low temperatures (“cold”), and total SO₂, i.e., of the originally dissolved and bound SO₂ at elevated temperatures (“warm”)).

The determination of the free SO₂ (SO₂ dissolved in the matrix) is preferably carried out under mild conditions where possible, so that the bound SO₂ is not released by the reagents, since this would distort the measurement. The reference procedure provides for cooling of the reagent/matrix mixture to preferably below 20° C., most preferably to approximately 10° C.

In the determination of the total content of dissolved and bound SO₂, the SO₂ is preferably bound through the addition of an adduct former in excess. Pyruvic acid or a salt thereof can advantageously be used as adduct former. The salt has the advantage that it is present in a solid state and in this form is sufficiently chemically stable. The pyruvic acid has a relatively high binding affinity and delivers stable bisulphite adducts. It is preferably used in the form of a solution. This solution lies pre-metered in the sample container. However, it can also be added by the user through a dropping pipette.

For better mixing of the sample upon supply of the acid reagent a sample container is preferably used which has, for example, as substrate an agar layer, possibly mixed with some pyruvic acid or a salt thereof, on its base. This layer liquefies under the influence of acid/heat and ensures mixing of the solution (for example wine as matrix). The pyruvic acid or the salt of pyruvic acid is added to this agar layer as a solution or in solid form and the sample is then added. If sulphuric acid H₂SO₄ is added as reagent then this would sink downwards because of its high density, whilst the sample, for example wine, would float to the top because of its lower density. The dissolution process of the substrate, however, brings about an advantageous mixing, so that sample and acid are sufficiently in contact, even at small sample volumes.

According to a further aspect of the invention a method is proposed for the determination of a content of at least one foreign substance in a matrix of a solid or liquid food, in which an originally dissolved foreign substance is first bound in the reaction area in the matrix, and foreign substance originally dissolved in the matrix is released in a delayed manner such that the originally dissolved foreign substance and any foreign substance originally bound are released together from the matrix in the same process step; and the determination of content of the foreign substance released in the same process step is carried out by a sensor.

An adduct former which binds the dissolved foreign substance can advantageously be added to delay the release of the foreign substance. A reagent can preferably be added for the release of the foreign substance.

Furthermore, the adduct former can advantageously contain pyruvic acid or a salt of pyruvic acid and the reagent can contain sulphuric acid and/or phosphoric acid.

The matrix can preferably be a liquid and/or a solid food, in particular wine or a wine constituent, fruit juices, beer, dried fruit, in particular an extract from dried fruit and the foreign substance can be SO₂ that is bound in food and/or dissolved.

Further, it is proposed to use one or more reagents and/or one or more adduct formers that are suitable for implementing a method for testing of solid and liquid foods, especially wine, fruit juices, beer, dried fruit and the like for one or more foreign substance contents.

### BRIEF DESCRIPTION OF THE DRAWINGS

The invention is described in more detail below with reference to the embodiments shown in the drawing. The Figures are shown schematically

FIG. 1 A preferred measurement arrangement of a device according to the invention;

FIG. 2 A perspective view of a preferred device according to the invention;

FIG. 3 A reagent container inserted in a reagent area of the device of FIG. 2, shown in detail;

FIG. 4 A sample holder connected to a reaction area shown in detail;

FIGS. 5a, 5b A preferred sensor (FIG. 5a) and a reference electrode (FIG. 5b) for the sensor;

FIG. 6a-6c A preferred ready-made sample container in different stages of preparation for determination; and

FIG. 7 A flow chart of a preferred measurement process.

Same or equivalent-acting elements are assigned the same reference numerals in the drawings.

### DETAILED DESCRIPTION

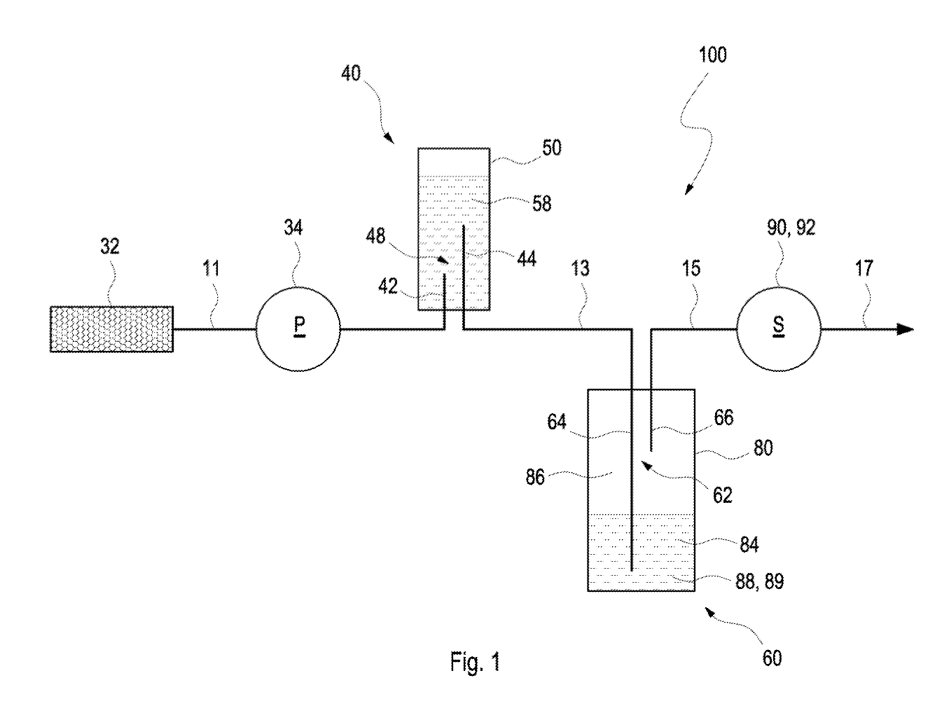

FIG. 1 shows, for description of an aspect of the invention, schematically the construction of a preferred device 100. A reagent container 50 is connected via a transfer line 13 in flow connection with a sample container 80. Downstream of the sample container 80 a sensor area 90 is provided which is connected to the sample container 80 via a line 15. An output line 17 leads outwards from the sensor area 90. A pump 34 transfers a carrier medium through the pump line 11 to the reagent area 40. The carrier medium is preferably a gas, for example ambient air, which is expediently drawn in through a filter 32. The carrier medium can also be taken where appropriate from a gas cylinder. The pump 34 can be arranged upstream of the reagent container 50 in the pump line 11 or downstream of the reagent container 50, for example in the transfer line 13.

A reagent 58, for example sulphuric acid (H₂SO₄), can be specifically fed from the reagent container 50 to the sample container 80, said reagent completely releasing the foreign substance 89 contained in a matrix 88 in unbound form and possibly in bound form. It is provided that a possibly unbound (dissolved) foreign substance is fully bound in advance, in particular through addition of an adduct former.

The reagent container 50 is attached to a needle system 48 which, for example, comprises two hollow needles 42 and 44. The hollow needles 42, 44 of the needle system 48 preferably project upwards and are parallel to each other. An embodiment, for example with coaxial hollow needles or needles with an inner hose is also possible. In a favourable further development, not shown, the hollow needles 42, 44 may be integrated as a component in a carrier body which has connections to the pump line 11 and to the transfer line 13. The needle system 48 can then be easily replaced.

The carrier medium enters the reagent container 50 via the hollow needle 42 on the inlet side. The reagent is withdrawn from the reagent container 50 via the hollow needle 44 on the outlet side and is fed to the sample container 80. A matrix 88 therein, which contains the foreign substance 89 to be detected, such as wine with a content of SO₂, is mixed with the reagent 58 and the content of foreign substance 89 is determined in the sensor area 90. A sensor 92 is provided in the sensor area 90 for determination. The foreign substance 89 is preferably passed in gas form into the sensor area 90 and fed to the sensor 92. The foreign substance 89 can also be fed to the sensor 92 as a solution where appropriate.

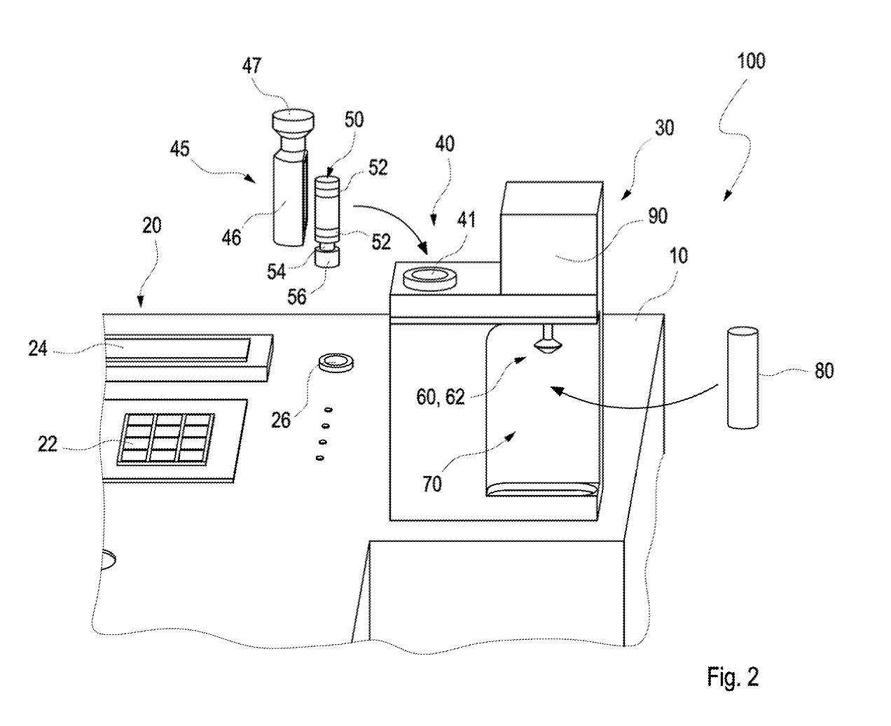

The reagent container 50 is preferably a pre-filled, in particular pre-dosed ampoule which the user simply inserts when needed in the device 100 described in more detail below. The reagent container 50 configured as a pre-dosed ampoule, contains one or more reagents 58 suitable and/or necessary for the specific detection of a foreign substance in exactly the dose that is matched to the quantity of sample (matrix 88 with foreign substance 89). The reagent container 50 is advantageously configured, i.e., its dimensions are such, that it can be introduced into a corresponding reagent area 40 (FIG. 2) that is configured in a complementary manner of the device 100. Incorrect operation the device 100 can thereby be avoided.

The reagent container 50 can be connected, for example in the form of a pierceable ampoule with a pierceable septum, as closure with a two needle system 48 of the device 100. The reagent container 50 can also be an ampoule, a vial with snap-on lid, a vial with crimp closure or the like.

The reagent container 50 is thereby attached to the needle system 48 through its under-side. The “upside down” assembly of the reagent container 50 enables it to be reliably emptied. An assembly is also conceivable, however, in which the reagent container 50 is connected to the reagent area 40 with the closure facing upwards or to the side.

The carrier medium, for example air, which is later required for measurement, transported by the pump 34, enters through the hollow needle 42 on the inlet side shown on the left in the figure, into the reagent container 50 in the form of an ampoule. Through excess pressure, the reagent 58 is transferred through the right hollow needle 44 on the outlet side into the further measurement system of the device 100. By means of the following carrier medium stream delivered by the pump 34, the foreign substance 89, for example SO₂, driven out of the matrix 88 by the reagent 58, is fed from the sample container 80 to the sensor 92 of the sensor area 90. The hollow needle 42 on the inlet side preferably extends into the reagent container 50 to a lesser extent than the hollow needle 44 on the outlet side, so that an excess pressure that may already be present in the reagent container 50, or which is generated upon piercing, is reduced through the hollow needle 44 on the output side before the hollow needle 42 on the inlet side comes into contact with the interior of the reagent container 50.

The enclosed volume of the reagent container 50 preferably lies between 0.2 ml and 15 ml, preferably between 0.5 and 10 ml. The enclosed volume of the reagent container 50 can be completely or partially filled with the reagent 58, for example a liquid. The reagent container 50 is preferably configured to hold a reagent quantity for exactly one measurement. A new reagent container 50 can therefore be used for each measurement. The fill quantities of reagent container 50 and sample container 80 are preferably matched exactly to one another. With particular advantage, the sample container 80 can already contain one or more specific chemicals in exactly measured quantities for the detection of the foreign substance 89. The reagent container 50 and the sample container 80 can preferably be made available as a detection-analysis set. The user only has to add to the sample container 80 a predetermined amount of the matrix 88, which contains the foreign substance 89. The user can therefore always carry out the determination of foreign substance content in the matrix 88 under precisely defined conditions.

Even in cases where the reagent 58 exhibits corrosive behaviour and/or is sensitive to air, moisture and/or other constituents of the surrounding room air at the point of use for detection or during transport and storage, stable measurement and certain transfer to the sample container 80 are possible. Outside the device 100, the reagent 58 in the reagent container 50 is tightly sealed until use and only after the attachment of the reagent container 50 to the needle system 48 as needed is it accessible. Moreover, the quantity of the reagent 58 is limited to that quantity required for detection and does not need to be metered by the user.

In a preferred use of an aspect of the invention, SO₂ is detected as a foreign substance 89 in wine as matrix 88. To detect the total content of dissolved and bound foreign substance, in particular SO₂ in wine, H₂SO₄ is preferably used as the reagent 58, in a concentration such that after mixing of the reagent 58 with the sample (including additions such as adduct former etc.) the acid concentration is preferably 15%-85%, preferably 40%-85%. Other organic or inorganic acids, for example phosphoric acid, can favourably be used in comparable concentration ranges.

The needle system 48, as well as the following downstream line 13 are preferably protected against corrosion. Stainless steel capillaries of stainless steel types that are available commercially can favourably be used for the needle system 48 and/or the line 13 and for the other lines 11, 15 and 17, for example stainless steels 1.4401, 1.4571, 1.4541, 1.4301, 1.4404, or alloys, in particular alloys containing Cr, Mo, Ni. It is also conceivable to use other materials such as precious metals, glass, ceramics, plastics.

Stainless steel can normally be severely attacked by the reagent 58 used, which contains, for example, semi-concentrated sulphuric acid, if it is in contact with it for a longer period. In accordance with an advantageous further development of an aspect of the invention, the reagent 58 is therefore mixed with an inhibitor that reduces or completely prevents corrosion. For the detection of SO₂, for example, a 40%-85% sulphuric acid is modified through the addition of one or more inhibitors, such as iron (III) salts, such that there is no longer an attack on the stainless steel capillary. The inhibitor is expediently added to the reagent 58 and can expediently be already contained in the pre-filled reagent container 50.

If different reagents 58 are used in the device 100 according to an aspect of the invention, for instance to detect the presence of a foreign substance 89 in the matrix 88 by different methods, then a suitable inhibitor is expediently added to all corresponding reagents 58, which are used in a reagent container 50, to maintain a defined inhibitor concentration. Such a case can occur, for example, when the device 100 contains “free” foreign substance 89, i.e., that is not bound in matrix 88, but instead is in solution and two different methods are to be used to detect the total content of foreign substance, namely free foreign substance and bound foreign substance in the matrix 88. For the determination of free and total foreign substance content, for example, two different reagents 58 are used in the same system, to which, for example, an appropriate amount of inhibitor, such as iron (III) salts, has been added.

If the inhibitor concentration is markedly reduced compared to the reagent 58, for example through inadequate rinsing of the capillary with pure water, then corrosion phenomena can occur. It is therefore recommended that the capillaries be rinsed sufficiently with pure water or other suitable rinsing solutions. Rinsing with an aqueous solution of the inhibitor can be advantageously carried out. This may be additionally, or alternatively, to the fact that the reagent 58 is mixed with the inhibitor.

After connecting the reagent container 50, the reagent 58 may be completely transferred through the transfer medium pumped by the pump 34 from the reagent container 50 into the sample container 80.

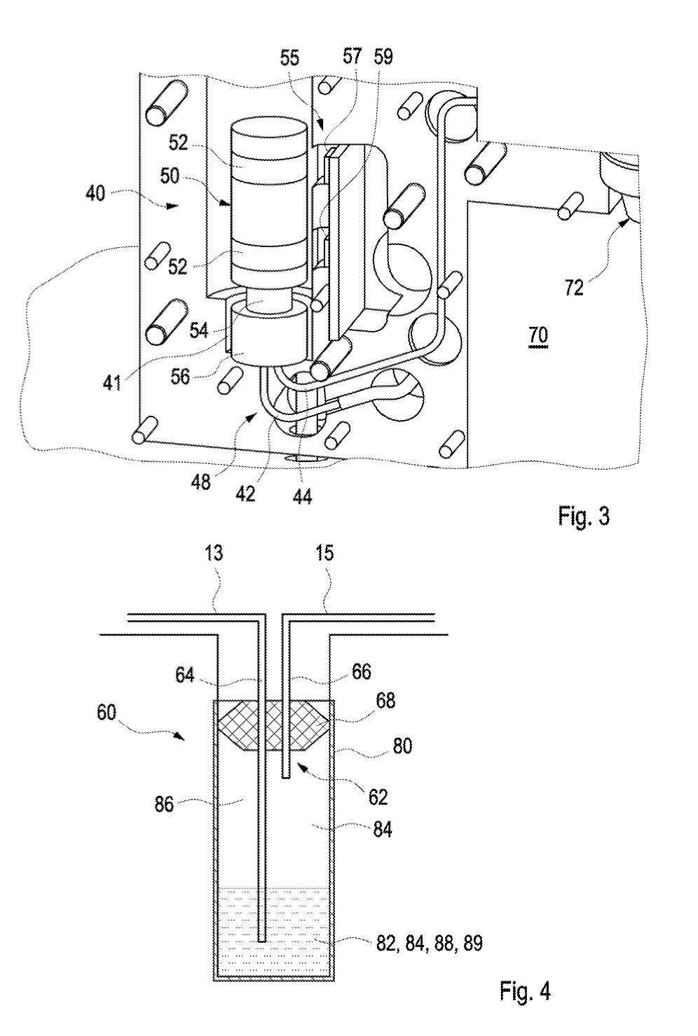

The sample container 80 is preferably configured so that it can be connected to a suitable reaction area 60 of the device 100. For this the reaction area 60 can have a cone-shaped coupling 72, that can have a sealing ring on its outer circumference, over which the, for example cylindrical, sample container is pushed. Instead of a sealing ring a press fit or other suitable sealing means can also be provided. The coupling 72 can be recognised in the recess 70 in the detail view of FIG. 3.

The sample container 80 preferably has dimensions such that when it is full exactly one measurement can be carried out for the detection of a foreign substance 89 in a matrix 88 with matched volumes of matrix 88 and an adduct former 84. The addition of adduct former 84 allows the simultaneous determination of the content of dissolved and bound foreign substance, especially of SO₂. The adduct former 84 is preferably contained in the pre-filled sample container 80 and does not need to be supplied by the user. Alternatively, the adduct former 84 can be separately provided to the user in a suitable dosage in the preferred detection analysis set and fed only at the time of measurement.

The reaction area 60 preferably comprises a connection system 62 with a first connection 64 on the inlet side through which the reagent 58 can be fed, and a second connection 66 on the outlet side, through which the foreign substance 89 can be passed to the sensor area 90. The connections 64 and 66 project out of the conical coupling, wherein at least one of the connections 64 passes through a stopper 68 (FIG. 4) that serves as a splash guard for the sample container 80. The connections 64, 66 can be formed as hollow needles, or be formed as hoses or small tubes. A coaxial embodiment with hollow needles that extend inside one another and/or hoses and/or small tubes is also possible.

If the sample container 80 is attached with its opening open in an upward direction to the coupling of the reaction area 60, then the connection 64 on the inlet side, which feeds the reagent 58, preferably extends deeper into the sample container 80 than the connection 66 on the outlet side, through which the foreign substance 89 to be detected or a reaction product thereof is fed out of the sample container 80 to the sensor area 90. The connection 64 extends in particular into a mixing area, in which the matrix 88, foreign substance 89 and, for detection of the total content of SO₂, the adduct former 84 are mixed, so that the reagent 58 from the connection 64 can come into intimate contact with the foreign substance 89 bound and/or free in the matrix 88. The foreign substance is released by action of the reagent 58 into the headspace 86 above the mixing area and can exit from the sample container 60 in the direction of the sensor area 90 through the connection 66 on the outlet side, which projects into the headspace 86.

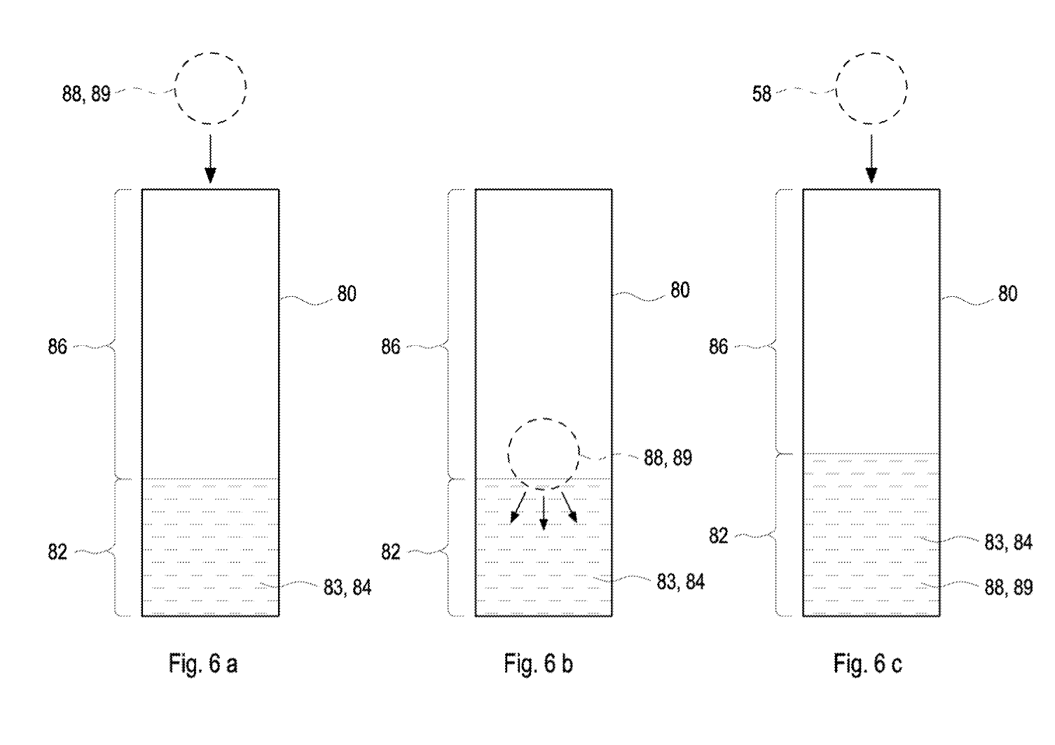

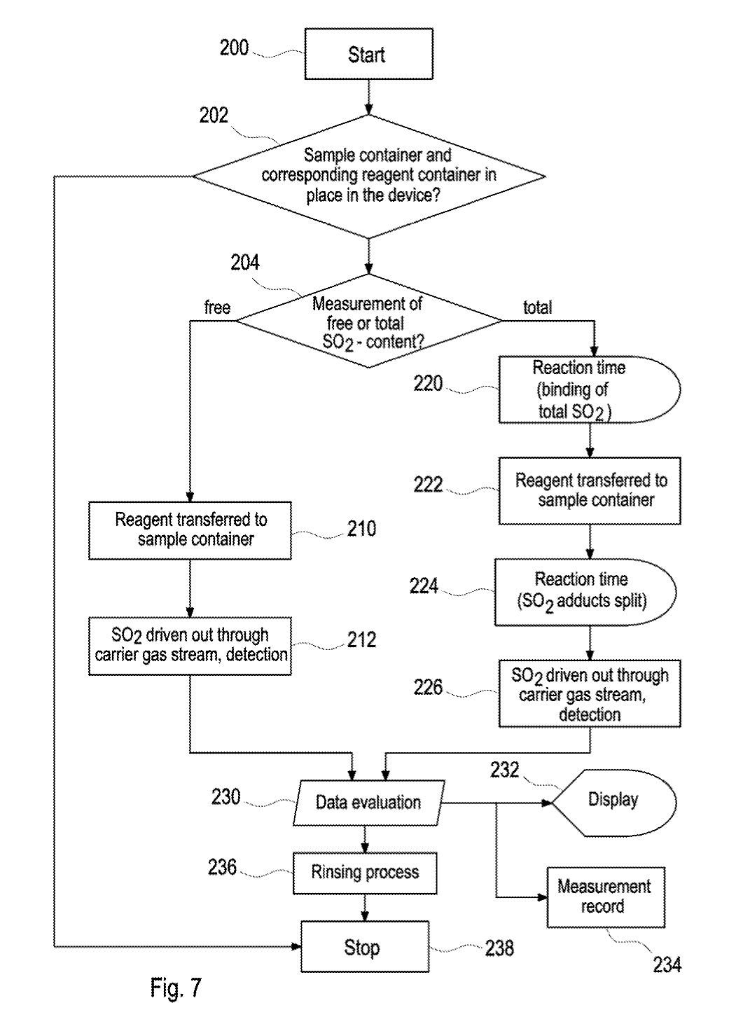

For the detection of foreign substance 89 it can be favourable to maintain the sample container 80 at a desired temperature or within a desired temperature range. The sample container 80 can where necessary be heated or cooled, depending on the reaction that is being carried out in the sample container 80 at the time. Further details of the detection sequence are described with reference to FIGS. 6 and 7.

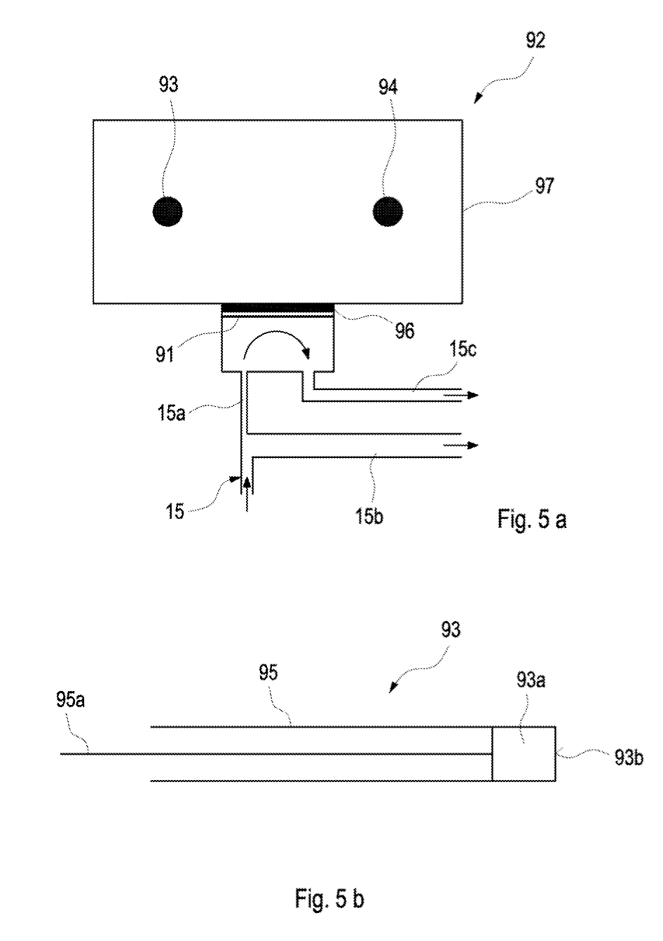

The foreign substance 89 released from the matrix 88 through the reagent 58 enters the sensor area 90 via the connection 66. The sensor area 90 preferably has an electrochemical sensor 92, which is explained in detail by reference to FIGS. 5a and 5b.

FIG. 2 shows, by way of example, a possible embodiment of a preferred device 100 for detecting a foreign substance in a matrix in accordance with the basic arrangement shown in FIG. 1. To avoid unnecessary repetition, reference is made to the components described therein, if they are not further explained here.

The device 100 is arranged in a housing 10 which houses a control part 20, a reagent area 40 with a receptacle 41, for example arranged as a depression, for the reagent container 50 and an analysis part 30 with the reaction area 60 for the sample container 80 and the sensor area 90. A line system comprising a pump line 11 and a transfer line 13 (FIG. 1) serve for transfer of the reagent 58 (FIG. 1) from the reagent container 50 into the sample container 80. The carrier medium stream can also be produced by a separate pump or a pressurized storage vessel and dosing be provided as a separate unit. Instead of the receptacle 41 for the reagent container 50, a metering pump and a reservoir vessel/another metering unit could be used, in principle, to feed the reagent 58.

The control part 20 comprises, for example, a keyboard 22, a display 24, a start button 26 and a corresponding processing unit (not shown) for control and if necessary for evaluation of the measurement and for monitoring of the device 100.

The device 100 is very compact and allows secure handling of reagent container 50 and sample container 80 with quantities and volumes of the chemical components used that are exactly matched to one another. The reagent container 50 can be used without risk of mix-up, and the device 100 in the reagent area 40 can have a sensor device 55, for example a light barrier 57, 59, a mechanical switch or the like, which can establish the presence of the reagent container 50. For this, the reagent container 50 can have reflective markings 52, through which the device can recognise the reagent container 50 used. Furthermore, as a simple coding this can enable recognition of reagent containers 50 for different reagents. However, other codings, for example bar code, mechanical coding, etc., can be used so that the device can reliably prevent measurements being carried out using reagents and samples not intended for one another. Operational reliability is thereby increased further, even with inexperienced users:

The receptacle 41 in the reagent area 40 is preferably formed as a depression into which the reagent container 50 can dip. The reagent container 50 can favourably be inserted into an insert 45 for this purpose. The reagent container 50 can preferably hold a volume between 0.2 ml and 15 ml, preferably between 0.5 ml and 10 ml, of the reagent 58.

A preferred embodiment of the reagent container 50 has a cylindrical cross section with a constriction 54 at one end, to which a closure 56, such as a pierceable septum, is connected. The insert 45 partially envelops the reagent container 50 through its body 46, wherein a region for recognition of the markings 52 remains free, and engages in the constriction 54 such that the reagent container 50 with the carrier 45 can be pressed into the receptacle 41 of the reagent area 40 in the form of a recess, to introduce the needle system 48 (FIG. 1) into the reagent container 50. The insert 45 has a retaining knob 47 at its upper end which projects out of the recess 41 upon attachment of the reagent container 50. This allows the reagent container 50 to be easily removed again from the device 100 after the measurement. The receptacle 60 is disposed in a recess 70 in which the sample container 80 is inserted and over which a cone of the reaction area 60 can be pushed, through which the connection system 62, for example a needle system, is accessible. A sensor can be arranged in the recess 70, for example a photocell, a micro-switch or the like, which recognizes the presence of the sample container 80 and passes this on to the control system of the device 100. If the sample container 80 is not inserted then the measurement cannot be started.

FIG. 3 shows the reagent area 40 of the device 100 of FIG. 2 with a reagent container 50 inserted in the recess 41 in a partly-exploded view. The reagent container 50 is attached with its closure 56 facing downwards to the needle system 48 with the hollow needles 42 and 44, as described in FIG. 1. The insert 45, which envelops the reagent container 50 is not shown for clarity reasons. A sensor 55, for example a light barrier with two transmitting and receiving units 57, 59, is arranged next to the reagent container 50 and detects the presence of the reagent container 50, for exampling through reflective markings 52 on the reagent container 50. If the reagent container 50 is not inserted, then the measurement cannot be started. A cone-shaped coupling 72 is recognisable in outline in an adjacent recess 70, onto which the sample container can be pushed (not shown).

FIG. 4 shows how the preferred sample container 80 can be connected to the reaction area 60. The reaction area 60 comprises a feed system 62 configured by way of example as a needle system, with a connection 64 on the inlet side configured as a hollow needle, through which the reagent 58 (FIG. 1) is fed and a connection 66 on the outlet side configured as a hollow needle, with which the carrier gas containing the foreign substance 89 is removed from the sample container 80. The feed system penetrates through a stopper 68 which serves as splash protection. The connection 64 on the inlet side as a hollow needle extends into a mixing area 82, in which the matrix 88, foreign substance 89 and an adduct former 84 are mixed, so that the reagent 58 from the connection 64, configured as a hollow needle, comes into intimate contact with the bound and/or free foreign substance 89 in the matrix 88. The connection 66 on the outlet side configured as a hollow needle projects into the headspace 86 above the mixing area 82. The connection 66 can end below or above the stopper 68 since this does not seal the sample container 80 in a gas-tight manner.

In an alternative arrangement the sensor 92 can be arranged in this headspace 86, instead of in a remotely-located sensor area 90. It is also conceivable to have an immersion sensor with a gas-permeable membrane that is immersed directly in the mixing area 82.

FIG. 5a shows schematically a three-electrode system with a reference electrode 93 as preferred electrochemical sensor 92, which is shown in detail in FIG. 5b. The reference electrode 93 serves as a potential reference point for the electrochemical measurement, which can be carried out either potentiostatically or galvanostatically.

The sensor 92 should preferably be operated with a chloride-free reference electrode 93. The possibilities afforded by such a usage, which is to be available with little effort and for relatively inexperienced users, are severely limited. An advantageous solution that is favourable in terms of environmental technology is a lead/lead sulphate electrode (Pb/PbSO₄), which, however, is described in the literature as only conditionally stable and therefore is regarded in practice as not usable.

This can, however, contrary to general opinion, be continuously regenerated in accordance with an aspect of the invention through a preferred electrochemical treatment so that a potential can be obtained that is sufficiently stable for the duration of analysis. Before each analysis, and in between each analysis, the reference electrode 93 is therefore preferably regenerated and then offers a stable potential for about 5 minutes. This period is entirely sufficient for a measurement procedure. The value holds for a so-'called micro-electrode. Increasing the surface area enables this time to be extended accordingly.

The advantage of the regenerative system, especially the Pb/PbSO₄ system, is evident in several aspects. The system of reference electrode 93 is chloride-free, the long-term stability of the system is more than a year in installed condition. Simple and non-critical handling is possible, and the reference electrode 93 is stable in an acidic environment. There is no “bleeding” of the system through differences in concentration, and there are no disturbances or poisoning of the working electrode 96 (SO₂-sensitive).

The reference electrode 93 has the theoretically-calculated potential directly after manufacture. After installation there is usually a marked drift, which can lead to a deviation of several hundred mV. As a result, this electrode would not be useful for analytical systems, where the maximum permitted deviation is just a few mV. According to the preferred electrochemical treatment according to an aspect of the invention, however, the reference electrode 93 can be regenerated in a few seconds before each measurement to exactly the starting potential.

A preferred electrochemical treatment can be carried out by applying an electrical voltage to the reference electrode 93, which enables current to flow. Typically, the potential of the reference electrode at the time of the regeneration process is approximately −1.5 to −3 volts relative to the counter electrode. The counter-electrode 94 in the electrochemical sensor 92 is therein used as a counter pole.

The working electrode 96 of the electrochemical sensor 92 is disposed on a porous membrane 91 and comes in contact with a carrier gas flowing past it, for example air, that is mixed with the foreign substance 89, for example SO₂ mixed with air, through the line 15 from the sample container 80 (FIG. 4). The carrier gas stream is indicated by arrows and is led away through line 15c from the membrane 91. The reference electrode 93, together with the auxiliary electrode 94, for example a gold wire, is housed within the sensor housing 97 in a conductive liquid. Preferably, the liquid contains sulphate ions (SO₄²⁻) the liquid is in particular preferably H₂SO₄.

To avoid drying out of the membrane 91, a large proportion of the carrier gas stream is routed past the membrane 91. To this end, the line 15 splits into a very narrow branch 15a, which is much narrower than the line 15, and into a wide branch 15b, which is much wider than the branch 15a. The branches 15b and 15c open separately into the line 17, through which the carrier gas stream can leave the sensor 92, but may also be merged beforehand. Alternatively, they can also be routed individually outwards.

In addition to a possible drying-out of the membrane 91, preferably a Teflon membrane, and/or the working electrode 96, which can be deposited as a layer on the membrane 91, splitting of the carrier gas stream in the branches 15c and 15b enables avoidance of a sensor signal that is too large, and allows compensation for fluctuations in the gas flow. This splitting can optionally also be designed to be switchable, so that the entire carrier gas stream with the foreign substance 89 flows via the sensor 93. Alternatively, the splitting can be replaced by a less gas-permeable sensor membrane.

In a preferred rinsing cycle to clean the sensor area 90/the sensor 92, the splitting process is switched off for about 95% of the rinsing time by closing the branch 15b, so that an increased rinsing action can be achieved.

The sensor 92 can be rinsed regularly with a moist medium to supply the membrane 91 with sufficient moisture.

The reference electrode 93 preferably comprises a Pb/PbSO₄ system and can have, as shown in FIG. 5b, a Pb inclusion 93a at the head end of a tube 95, for example a glass tube, which projects outwards with a Pb surface 93b. The Pb inclusion 93a seals the inside of the tube 95 against penetrating liquid. The bond wire 95a serves for electrical contact to the Pb inclusion 93a in the regeneration of the reference electrode 93.

If the reference electrode 93 is formed from a lead/lead sulphate system (Pb/PbₙXₘ system), then the reference electrode 93 can be regenerated in its properties through an electrochemically-induced reaction. Upon contact with the conductive liquid the sulphate PbSO₄ is formed from the metallic Pb and upon regeneration metallic Pb is formed from PbSO₄. X preferably represents a particular ionic species in the conductive liquid. In particular, the Pb/PbₙXₘ system is a Pb/PbSO₄ system with n=1, m=1 and X=SO₄²⁻. The electrochemically active materials in a Pb/PbSO₄ system are Pb and Pb (II) sulphate and/or Pb and Pb (IV) sulphate. A reference electrode 93 in which SO₄²″ is replaced by PbO is also conceivable.

In principle, such a reference electrode 93 in the form of reference electrode 93 can also be used in other sensors, such as biosensors, chemical sensors or the like in which a chloride-free reference electrode system is desired. An improvement in stability can be achieved through the use of alloys instead of a pure metal, as for example here Pb.

Instead of an electrochemical sensor 92 another method with a different sensor type can naturally also be used and, for instance, a photometric determination of the foreign substance 89 can be carried out. The electrochemical sensor 92 is therein replaced, for example, by a photometric detector unit (not shown). In the case of detection of SO₂ in wine, by way of example, an indicator solution (pararosaniline formaldehyde mixture/DTNB) or sulphite oxidase and NADH can be introduced into the sample container 80, and the blank value determined photometrically. SO₂ is then released as described later, wherein a reaction (colour reaction or consumption of NADH) takes place, which can be detected and quantified photometrically. For coloured samples, it may be favourable to add the indicator solution or to run the enzymatic determination not in the sample, but by separating the sample and the indicator through a semi-permeable membrane, a gas stream or the like. With less coloured samples determination can be carried out directly in the sample.

The released SO₂ is combined with the pararosaniline formaldehyde mixture, and a colour reaction is seen after a corresponding waiting period. A photometric unit can be used to measure and quantify the absorption change at a specific wavelength. Another possibility is a colour reaction with DTNB (5,5′-dithio-bis(2-nitrobenzoic acid)). In the presence of sulphite DTNB is cleaved to TNB (2-nitro-5-mercaptobenzoic acid), which causes an intense yellow coloration, which can be quantified photometrically. Similarly, an enzymatic detection can be used. Sulphite oxidase is used in the enzymatic detection. In the presence of oxygen and water, sulphate and hydrogen peroxide are formed from sulphite, which in turn reacts with NADH (nicontinamide-adenine dinucleotide). The quantitative determination is carried out photometrically via the consumption of NADH. The detector unit can thereby be separated from the sample through a membrane or a gas line system.

FIGS. 6a-6c illustrate the preparation of a preferred sample container 80 for determining the content of a foreign substance 89 in a matrix 88, in particular a determination of SO₂ content in wine, in particular the entire content of dissolved and bound SO₂.

In accordance with a preferred embodiment, the pre-filled sample container 80 is filled with a substrate 83 that is stored in the bottom area of the sample container 80 (FIG. 6a), which later forms a mixing area 82. The substrate 83 is, for example, a gel, gelatine, agar-agar or the like, which dissolves upon contact with water, causing vortexing of the solution and thus good mixing of the components coming into contact therewith, even with relatively small sample volumes. An adduct former 84 is preferably added to the substrate 83. It is not necessary to use the substrate 83.

An adduct former is added to bind the content of dissolved foreign substance 89. The adduct former 84 is a binding partner for the dissolved foreign substance 89. Preferably, the entire “free”, i.e., dissolved foreign substance 89 is bound to the binding partner, through which the release of foreign substance 89 from the matrix 88 is influenced, in particular, is delayed. The release characteristics for the originally dissolved and originally bound foreign substance 89 are equalised. An acidic pH is preferably set for the release of the foreign substance 89.

In the determination of SO₂ in wine, a binding partner that has at least one aldehyde and/or ketonic group, enabled for bisulphite addition is preferably added immediately before the measurement to bind the remaining free SO₂ and to equalise the concentration and/or time profiles. This is done preferably when the total content of foreign substance 89 in dissolved and bound form is to be determined. If only the content of dissolved foreign substance 89 is to be determined, then addition of the adduct former 84 can be omitted.

The adduct former 84 can be added in solid form or in liquid form as a pure substance or solution. Advantageously, the adduct former 84 can be dosed as a solution in sample vials or freeze-dried and can therefore be transferred into a transportable state. An amount of adduct former 84 can be provided in sample containers 80 in ready-to-use form for the user that is matched exactly to the quantity of matrix 88 containing foreign substance 89 that is to be added.

Alternatively, for delayed release of the dissolved content of foreign substance a covering through a barrier layer is also conceivable, for example a floating layer with a lower specific density than water, for example an oil layer, which prevents premature release of the dissolved foreign substance 89. To avoid strong foam formation a defoamer may be added, such as a silicone oil-containing agent. Similarly, a delay of the foreign substance transfer can also be enabled through the formation of polymer in the sample, through addition of a polymer-forming component which polymerises preferably under acid influence. It is also conceivable to pass the carrier gas not through the matrix 88, but to arrange a permeable layer between the carrier gas and the matrix 88, which separates the carrier gas from the matrix 88 and permits the passage of the foreign substance 89 from the matrix 88 into the carrier gas in the headspace 86.

As indicated in FIG. 6a, a defined quantity of the matrix 88 under investigation, which possibly contains foreign substance 89, is added, for example using a pipette (FIG. 6b). The amounts of adduct former 84 and matrix 88 are advantageously matched to one another. The adduct former 84 is present in excess compared to the expected amount of foreign substance 89.

The matrix 88 containing the foreign substance 89 mixes with any substrate 83 present and the adduct former 84 (FIG. 6b), wherein the substrate 83 is dissolved and the foreign substance-containing matrix 88 and adduct former 84 are mixed. In accordance with an aspect of the invention the adduct former 84 first brings about binding of the free fraction of the foreign substance 89, which is dissolved in the matrix 88 (FIG. 6b). The originally free fraction of the foreign substance 89 is then in bound form and can be released together with fraction of the foreign substance 89 originally bound in the matrix as soon as the matrix 88 comes into contact with the reagent 58 (FIG. 6c).

The addition of the reagent 58 causes cleavage of the bonds, as a result of which the total content of foreign substance 89, i.e., the content originally dissolved in the matrix 88 and the content originally bound in the matrix 88, is released and can be transported out of the headspace 86 through a carrier gas to the sensor area 90. The delayed release of the originally dissolved content of the foreign substance 89 at the same time as the originally bound content of the foreign substance 89 enables measurement of the total content of foreign substance 89 in the matrix 88 with sufficient accuracy. Different concentration profiles for foreign substances 89 that are bound to differing degrees because of the measurement of foreign substance content not in chemical equilibrium can be advantageously avoided.

Thus, in the detection of SO₂ in wine, different concentration profiles of the sulphite-containing species, bound to differing degrees, are to be expected. Without the preferred measurement sequence according to an aspect of the invention, an early rise in the concentration of the sulphite, present as free SO₂, would be observed, which would then fall relatively steeply after reaching the maximum concentration. For species for which the chemical equilibrium lies strongly on the side of the bound sulphite, concentration curves can be seen that only reach a maximum value late on and then fall relatively slowly. In accordance with an aspect of the invention in the presence of very different species in unknown concentrations an equalisation of the concentration profile can be carried out for simple mathematical determination of the concentration via the sum of all sulphite species.

The use of a strong acid as a reagent 58, preferably at least half-concentrated sulphuric acid, is favourable for determination of foreign substance content for release of the bound foreign substance 89.

For this, the sample container 80 can be adjusted to a suitable temperature. A heating and/or cooling system can be provided (not shown) with a heating device and/or a cooling device as well as one or a plurality of suitable mounted temperature sensors. Alternatively or additionally, a substance can be added to the sample container which does not disturb the reaction, but which, however, has a suitable positive or negative enthalpy of solution. Negative enthalpy of solution means that when the substance is dissolved in a solvent, for example water, heat is released and positive enthalpy of solution means that the substance absorbs heat when it is dissolved. Thus the content of the sample container 80 can be selectively heated or selectively cooled without the need to provide additional mechanical or electrical components. The solvent can be advantageously contained in the reagent 58 and/or in the matrix 88.

For heating, the hydration energy of an acid can, for example, be specifically used, and it can be added, for example, as reagent 58. This allows the hydration energy of H₂SO₄ as reagent 58 to be advantageously exploited for the release of the SO₂. The H₂SO₄ is preferably used at least half-concentrated. This is particularly advantageous if the detection is to be carried out not only for dissolved, but also for the bound foreign substance 89.

Ammonium sulphate can advantageously be added for cooling as it withdraws heat when it is dissolved. This is advantageous if only the content of the foreign substance 89 dissolved in the matrix 88 is to be determined, but not the content of bound foreign substance 89. Detection of the dissolved foreign substance 89 in the matrix 88 is carried out under milder conditions than those for detection of foreign substance 89 bound in the matrix 88.

In addition to the elimination of the need for mechanical and/or electrical components as compared with known devices it is possible with the method according to an aspect of the invention to use a common line route for reagents that allow free or bound foreign substance 89 to be determined.

Heating of the sample container 80 is preferably carried out when the total content of foreign substance 89 in dissolved and bound form is to be determined. If only the content of dissolved foreign substance 89 is to be determined, then cooling of the sample container 80 is preferred.

A preferred procedure for measurement is described in more detail in FIG. 7 based on a determination of a sulphite content (SO₂, foreign substance 89) in wine (matrix 88).

The foreign substance content in the matrix to be investigated, for example a wine sample, can vary widely. A fraction may be present as free SO₂, which is dissolved in the wine matrix. A significant fraction, however, is bound to various components in wine (bisulphite addition to, for example, aldehyde or ketonic groups). Different components bind the SO₂ to varying degrees.

Digestion of the bound SO₂ is by means of a non-volatile acid. Phosphoric acid or sulphuric acid, for example, are favourable. The heat generation upon dilution is much more pronounced for sulphuric acid so that it alone can bring about complete conversion of the bisulphite adducts without any additional heat source. For example, an approximately 80% sulphuric acid can be used. For example, 1.5 ml acid can be added to 0.2 ml sample. Preferably, the acid is contained in a reagent container 50 in the form of a pre-dosed ampoule with pierceable septum, to avoid expensive dosing systems.

The corrosive action of sulphuric acid on most commonly-used stainless steels is disadvantageous, so that either inert materials are preferred for the line system in the device 100 and/or the corrosive action of acid is reduced by suitable additives. This is favourably achieved, for example, by the addition of iron salts to the acid. The choice of material is severely limited by the technical arrangement of reagent feed (e.g., pierceable ampoule) as this requires an adequate stability.

The free SO₂ in the wine, however, is determined under “mild” reaction conditions. The existing bisulphite adducts should not thereby be adversely affected. An acidic environment is required to drive the SO₂ out of the solution. A non-volatile acid is preferably used as acid (for example phosphoric acid). The reaction conditions can be optimized further by cooling the system, for example, by the addition of salts with a positive enthalpy of solution, which cause a cooling upon dissolution, or an active cooling through a suitable cooling element.

The measurement routine commences at step 200. In step 202 it is established whether the sample container 80 has been placed in the reaction area 60 and the corresponding reagent container 50 in the reagent area 40 and the device 100 (FIG. 1). The reagent container 50 is advantageously filled ready for use with the reagent 58 and only has to be placed by the user in the reagent area 40. The sample container 80 is preferably filled ready for use as necessary and is provided by the user shortly before the start of measurement with a defined amount of a wine sample to be investigated. By appropriate coding on the reagent container 50 and/or the sample container 80 it can be determined whether the reagent container 50 containing the correct reagent 58 is available for the test sample in the sample container 80.

If the reagent container 50 and/or the sample container 80 are not at the intended place in the device 100, or a false reagent container 50 has been inserted then the measurement routine cannot be started and it ends at step 238. Optionally, an alarm signal is issued and the malfunction is displayed to the user. The measurement routine can be started, for example, through a start signal by pressing a button, keyboard input, input via touch screen or the like. The start signal may be triggered before or after adjusting the reagent container 50 and the sample container 80.

If the reagent container 50 and the sample container 80 are correctly placed in the device 100, it is established in step 204, in which mode measurement is to be carried out, i.e., if only the free SO₂, is to be determined or if the total amount of free and bound SO₂ is to be determined in the wine.

If “total SO₂” is to be measured then an adduct former 84 is contained in the ready to use pre-filled sample container 80. This can be provided ready for use, with or without substrate 83, in the sample container 80 before the wine sample to be tested is added. Shortly before the start of measurement a wine sample is added in a defined amount to the sample container 80. During a short reaction time of, for example, 0.5-2 minutes, the free SO₂ contained in the wine is bound by the adduct former 84, for example a sodium salt of

pyruvic acid. The SO₂ fractions in the wine sample are now present only in bound form.

After sufficient reaction time, in step 222 the reagent 58, preferably H₂SO₄, is introduced into the sample container 80, whereby a carrier agent, for example the pump 34, pumps a carrier gas into the reagent container 50 and the reagent 58 is forced out of the reagent container 50 into the sample container 80.

After a reaction time of, for example, 1 minute, during which the SO₂ adducts are split into the educt SO₂ and adduct former 84, the released SO₂, which now contains both the originally dissolved and bound fractions, is driven out of the sample container 80 by a carrier gas stream, for example air, delivered by the pump 34 and is fed to the sensor area 90 (FIG. 2). A salt of pyruvic acid is preferably used as adduct former 84, and has the advantage that it is present in solid state and in this form is sufficiently chemically stable. The pyruvic acid has a relatively high binding affinity and delivers stable bisulphite adducts. The adduct former 84 can be added as a tablet or in liquid form as solution or liquid to the wine or be already contained in the sample container 80 in the desired quantity.

The data is evaluated in step 230, and is represented visually in a display in step 232, for example optically, with output of a measurement record in step 234.

If only the amount of free SO₂ is to be measured then the first reaction period in which the free SO₂ is to be bound is eliminated since no adduct former 84 was introduced into the sample container 80/a pre-prepared sample container was used without adduct former 84. Step 204 is therefore followed by step 210 in which the reagent 58 is transferred to the sample container 80. In step 212, a carrier gas stream flowing through the reaction mixture drives the SO₂ out and transports it to the sensor unit 90. The detection of free (dissolved) SO₂ requires only relatively mild reaction conditions. If necessary, the sample container 80 can be cooled to avoid an unwanted release of the bound SO₂ fraction in the wine sample because of an undesirable rising temperature.

The data is evaluated in step 230, and can be represented visually in a display in step 232, with output of a measurement record in step 234.

The analysis can theoretically be carried out in a number of ways:
Numerical integration of the measurement curve (area of the curve)
Average value over a certain range
Median over a certain range
Peak height
Optional baseline correction
Evaluation of the complete measurement curve up to a certain residual SO₂ content in the carrier gas
Evaluation of only parts of the measurement curve (time sections or dependence on signal levels/relative signal levels)
Evaluation of the signal plateau of the measurement curve (for example with a gas circulation in which the gas transported out of the sample container 80 is circulated to the sensor 92 and via the reagent container 50 back to the sample container 80 and sensor 92).

At the end of data evaluation the sample container 80 is removed from the device 100 and the sample head cleaned, for example, wiped with a damp cloth. A rinse vial can be inserted instead of the sample container 80, whereby the rinsing process is started automatically in step 236 and lasts, for example, 1 minute and the measurement routine ends in step 238. The rinse vial is then removed and the sample head wiped dry. The empty reagent container 50 is removed from the device.

Favourable parameters for the measurement are given by way of example below. Possible acids for the determination of SO₂ are sulphuric acid, phosphoric acid, hydrochloric acid as well as any organic or inorganic acids, which allow a suitable pH to be achieved. Acids which are not volatile, or have only a low volatility or if they are volatile do not interfere with the detection of SO₂ in the concentration range and temperature range used are preferably suitable.

A favourable pH range according to an aspect of the invention for the determination of total SO₂ content, i.e., the content of dissolved (“free”) and bound SO₂ is pH<1 for the solution, which is present in the sample container 80 after addition of the reagent 58. This can be achieved, for example, through approximately 0.1 mole/L sulphuric acid in this solution, or through approximately eight percent phosphoric acid in this solution, depending on the chemical composition of the solution.

The higher the degree to which the pH value is lowered, i.e., the higher the acid concentration in the solution, the more readily the bound SO₂ is converted into dissolved SO₂.

A favourable pH range for the determination of the content of free SO₂ is between 0.5 and 2, preferably between 0.8 and 1.5 for the solution which is present in the sample container 80 after the supply of reagent 58. This corresponds roughly to a concentration of 0.03-to 0.2 moles/L sulphuric acid in this solution. Accordingly, phosphoric acid can also be used in appropriate concentration.

The protolysis range of sulphurous acid is known to be such that at low pH values (for example pH=0) and high pH values (for example pH=10), the SO₂ is present almost entirely in the free form. To ensure complete release of the bound SO₂, the entire SO₂ should therefore be determined in the highly acidic region. Alternatively, determination is also possible in the highly alkaline range.

To obtain the molecular SO₂ present in the solution, the pH value should be below approximately pH=1.5, since only this form can be converted into the gas phase. To determine the content of dissolved SO₂ under these conditions it is necessary to counteract conversion of bound SO₂. For this a low temperature is preferably chosen to avoid a back-reaction of bisulphite adducts. This also makes it possible to use low pH ranges, preferably below pH=1.5, to obtain a presence of molecular SO₂ in the solution. A temperature between 0° C. and 25° C. is therefore suitable for determination of the content of dissolved SO₂.

To determine the total content of SO₂ in the wine matrix, the pH should be as low as possible. This firstly has a favourable effect on the presence of molecular SO₂ and secondly enables a back-reaction of the bisulphite addition. The pH value should be less than pH 1, and be preferably <0.5 for the detection of the bound SO₂. An elevated temperature can accelerate the back-reaction. Elevated temperatures and a strongly acidic environment are therefore favourable for the determination of the total content of SO₂.

Some preferred reagent compositions and quantities are given below. The percentages refer to percent by weight.

1) Determination of the Total Content of SO₂ in Wine:

Sample volume of wine in the sample container 80, for example 100-300 μl, preferably 150-250 μl.

Component 1: Adduct former (10-100 μl, preferably 20-50 μl per sample)

Concentrated solution of sodium salt of pyruvic acid in water.
A concentrated solution is favourable, since this takes up only a small volume. However, a more dilute solution can also be used.

Component 2: agar layer (100-400 μl, preferably 150-250 μl per sample)

0.3-2.5%, preferably 0.5-1.5% agar (solution of 0.3-2.5 g, preferably 0.5-1.5 g agar in 100 ml water)
5% sodium salt of pyruvic acid

Less than 0.3% agar leads to gels of low stability, too high a concentration leads to solid gels, which are less suitable. Preferred is a range of 0.3 to 2.5%. The pyruvic acid or salt thereof can optionally also be omitted, since the adduct former (component 1) is added and this addition cannot be replaced by the content in the gel.

Component 3: Acid (1-3 ml, preferably 1.25-1.75 ml/sample)

70-90% sulphuric acid with approx. 0.2 to 5%, preferably 1-1.5% Fe (III) sulphate hydrate. Acid concentrations that are too high present technical difficulties and can also influence the constituents of the wine in a disruptive manner. Concentrations below 60% require additional heating. With additional heating approximately 40% sulphuric acid can be used. The dilution by the quantity of wine (here 1.5 ml reagent and 200 μl wine) and other added reagents must also expediently be taken into consideration.

The quantity of sample can also be increased, whereby the quantities of at least the components 1 and 3 are expediently increased in the same ratio to the changed sample quantity.

2) Determination of the Content of Free SO₂ in Wine:

Sample volume of wine in the sample container 80, for example 100-300 μl, preferably 150-250 μl

Component 1: Acid (1-3 ml, preferably 1.25-1.75 ml/sample)

5-20%, preferably 10-15% phosphoric acid with approximately 0.2-5%, preferably 0.6-0.8% Fe (III) sulphate hydrate

Higher acid concentrations release too much bound SO₂ and concentrations that are too low yield values that are too low compared to standard reference methods. The preferred acid concentration is in the range 5% to 20%. It is also necessary here to take the dilution by the quantity of wine (here: 1.5 ml reagent and 200 μl of wine) and possible additional reagents into consideration. The iron salt is here added only for the reason that the same line system in the device 100 is used for both variants (total SO₂ content, content of free SO₂), and thus a certain corrosion-inhibiting iron content in the device 100 is assured.

Component 2: (optional) ammonium sulphate (100-500 mg, preferably 150-250 mg/sample)

A salt, which withdraws heat during the dissolution process can optionally be added, preferably a salt which does not release any volatile components in combination with acid. Preferred is ammonium sulphate. If ammonium chloride is used then hydrochloric acid formed therefrom can be driven out in small quantities by the carrier gas, and if ammonium nitrate is used then disruptive nitrogen oxides can be formed.

Where possible, so much is added that an undissolved residue remains. This leads to a nearly saturated solution being obtained with reproducible results. Any errors in the weighing out of quantities will not play such a large role here. Lower quantities can naturally also be used, but in this case the weighing-out should be quantitative.

The sample (matrix 88 with foreign substance 89) can be preferably supplied by a metering pipette, for example a piston-driven pipette. Numerous other variants are, however, conceivable, such as metering pumps etc. Furthermore, a dilution of the sample in the system is also conceivable, for instance by supplying water or dilute acid etc., possibly also in heated form, possibly also superheated steam as the carrier gas.

The software used allows detection of possible deviation from “normal conditions”. For example, a deviation of the zero line is registered, which can mean a contamination of the outside air or of the system. A carbonate-coated activated charcoal filter can be used, for example, against contaminated outside air. Purely mathematically, the basic content of the ambient air can naturally also be deducted from the measurement result, so that there is sufficient correction.

Moreover, the control unit of the device 100 has safety features, which, for example, bring about disabling of the pump 34 upon incorrect operation or compare the bar code for the reagents used for conformance with the selected method.

An error analysis can be carried out for the measurement curve within certain limits, so that the reliability of the measurement results is increased. The sensitivity of the sensor 92 can also be checked so that the condition of the sensor 92 is determined.

The invention, according to an aspect thereof, advantageously enables simple handling through pre-dosed components and a quick analysis through short reaction times. Heating through the reagent dispenses with the need for expensive components such as a heating block. The hollow needles of the needle system in the reagent area have the necessary hardness to enable use in conjunction with a pierceable septum. Since such systems are frequently used in other areas, these are available as low cost materials. A line system of inert materials, such as

Teflon or other plastics, glass, ceramics and the like is, however, also conceivable.

1. Device (100) for determining a content of at least one foreign substance (89) in a matrix (88) of a solid or liquid food, characterised by at least
one reagent area (40) for providing at least one reagent (58) with a receptacle (41) for an interchangeable reagent container (50);
one reaction area (60) with a connection system (62) for interchangeable connection of a sample container (80) at least containing the matrix (88);
one transfer line (13) between the reagent area (40) and the reaction area (60), with which the at least one reagent (58) can be transferred to the reaction area (60);
one sensor area (90) for the detection of the foreign substance (89) released from the matrix (88);
one carrier gas line (15) between the reaction area (60) and the sensor area (90), through which the released foreign substance (89) can be transferred to the sensor area (90); and
one output line (17) through which the dissolved foreign substance (89) can be transferred from the sensor area (90) outwards.

2. Device according to claim 1, characterised in that the reagent area (40) comprises at least one sensor device (55) which automatically detects a presence or absence of the at least one reagent (58) and/or reagent container (50) in the reagent area (40).

3. Device according to claim 1 or 2, characterised in that the reagent area (40) has a receptacle (41) which accommodates the one reagent container (50).

4. Device according to any one of the above claims, characterised in that for connection of the reagent container (50) the reagent area (40) has a needle system (48) to which the reagent container (50) is attachable, in particular wherein the needle system (48) has at least one input-side hollow needle (42) and one output-side hollow needle (44) or a hollow needle with an inner hose.

5. Device according to any one of the above claims, characterised in that, for connecting a sample container (80), the reaction area (60) has a feed system (62), through which at least the reagent (58) can be fed and released foreign substance (89) can be discharged.

6. Device according to any one of the above claims, characterised in that a sensor can be inserted into a headspace (86) of the sample container (80).

7. Device according to any one of the above claims, characterised in that the reaction area (60) has a heating and/or cooling device.

8. Device according to any one of the above claims, characterised in that the sensor area (90) houses an electrochemical sensor (92), in particular wherein the electrochemical sensor (92) comprises a reference electrode (93) with a chloride-free redox system, preferably Pb/PbSO₄.

9. Device according to any one of the above claims, characterised in that the sensor area (90) comprises a photometric sensor.

10. Device according to any one of the above claims, characterised in that the carrier gas line (15) between the reaction area (60) and sensor area (90) is divided into a first branch line (15a) for supplying carrier gas to the sensor area (90) and a second branch line (15b) for bypassing the sensor area (90).

11. Interchangeable reagent container (50) for a device (100) for determining a content of foreign substance (89) in a matrix (88) according to any one of the above claims, which is prepared ready for use with a defined quantity of at least one reagent (58), and which is interchangeably connectable to the receptacle (42) of the reagent area (40).

12. Reagent container according to claim 11, characterised in that one or more codings (52) are provided to detect the presence in the device (100) of the contained reagent (58) and/or its nature.

13. Reagent container according to claim 11 or 12, characterised in that in addition to the reagent (58), a corrosion inhibitor is contained.

14. Interchangeable sample container (80) for a device for determining a content of foreign substance (89) in a matrix (88) according to any one of claims 1 to 12, which is prepared ready for use and can be interchangeably connected to the connection system (62) of the reaction area (60), wherein at least one member of the group contained is
(a) an adduct former (84) in metered quantity that is matched to the foreign substance (89) to be detected and which serves for binding of any foreign substance (89) dissolved in the matrix (88);
(b) a substrate (83), which dissolves in contact with a reagent (58) and/or a matrix (88)
(c) a chemical component with a positive enthalpy of solution, so that the chemical component absorbs heat when dissolving in the matrix, in particular wherein the adduct former (84) is pyruvic acid or a salt of pyruvic acid, and/or in particular wherein the reagent (84) comprises sulphuric acid and/or phosphoric acid.

15. Sample container according to claim 14, characterised in that the substrate (83) comprises a substance that is capable of forming a network, in particular a three-dimensional network, preferably agar and/or gelatine.

16. Set, comprising a reagent container (50) according to any one of claims 11 to 13 and a sample container (80) according to any one of claims 14 to 15 for use in a device (100) for determining a content of at least one foreign substance (89) in a matrix (88).

17. Method for operating a device (100) for determining a content of at least one foreign substance (89) in a matrix (88) of a solid or liquid food, according to any one of claims 1 to 10, characterised in that
an originally dissolved foreign substance (89) in the reaction area (60) in the matrix (88) is first bound and
the foreign substance (89) originally dissolved in the matrix (89) is released with a delay such that the originally dissolved foreign substance (89) and any originally bound foreign substance (89) are released from the matrix together in the same process step.

18. Method according to claim 17, characterised in that for delayed release the matrix (88) containing the foreign substance (89) is mixed with an adduct former (84) to bind dissolved foreign substance (89) and that after binding of the originally dissolved foreign substance (89), a reagent is added, which drives the originally dissolved foreign substance (89) and any foreign substance originally bound (89) out of the matrix (88).

19. Method according to claim 18 or 19, characterised in that for delayed release the matrix (88) containing the foreign substance (89) is covered and/or a polymerisation is carried out, in particular wherein the driven-out foreign substance (89) is fed by a carrier gas to a sensor (92) for detection of the foreign substance (89).

20. Method for determining a content of at least one foreign substance (89) in a matrix (88) of a solid or liquid food, characterized in that
an originally dissolved foreign substance (89) in a matrix (88) is first bound and
a foreign substance (89) originally dissolved in the matrix (89) is released with a delay such that the originally dissolved foreign substance (89) and any originally bound foreign substance (89) are released from the matrix together in the same process step and
the determination of the content of the foreign substance (89) released in the same process step is carried out through a sensor (92).

21. Method according to claim 20, characterised in that to delay the release of foreign substance (89) an adduct former (84) is added that binds the dissolved foreign substance (89).

22. Method according to claim 20 or 21, characterised in that a reagent (58) is added for release of the foreign substance (89).

23. Method according to claim 21 or 22, characterised in that the adduct former (84) contains pyruvic acid or a salt of pyruvic acid and the reagent contains sulphuric acid and/or phosphoric acid.

24. Method according to claims 20 to 23, characterised in that the matrix (88) is a liquid and/or solid food, in particular wine, fruit juice, beer or dried fruits, and the foreign substance (89) is bound and/or dissolved SO₂.

25. Use of one or more reagents (58) and/or one or more adduct formers (84) which are suitable for carrying out a method according to any one of claims 20 to 24 for testing of liquid and/or solid foods, in particular wine, fruit juice, beer, dried fruit.
